(19) United States
(12) Patent Application Publication
(10) Pub. No.: US 2009/0059359 A1
(43) Pub. Date: Mar. 5, 2009

(54) SECONDARY LIGHT SOURCE

(75) Inventors: Werner Nahm, Buhlerzell (DE); Holger Matz, Unterschneidheim (DE); Markus Bausewein, Aalen (DE); Peter Reimer, Ellwangen (DE)

Correspondence Address: CASELLA & HESPOS, 274 MADISON AVENUE, NEW YORK, NY, 10016, US

(73) Assignee: Carl Zeiss Surgical GmbH, Oberkochen (DE)

(21) Appl. No.: 12/198,965

(22) Filed: Aug. 27, 2008

(30) Foreign Application Priority Data
Aug. 28, 2007 (DE) 10 2007 041 439.2

(51) Int. Cl. G02B 21/00; A61B 1/06

(52) U.S. Cl. 359/368; 362/574

(57) ABSTRACT

A secondary light source is provided comprising a narrowband light source (11) that emits narrowband light as a primary light source, an optical waveguide (5) having a proximal and a distal end (9), a coupling-in device (13) that is arranged at the proximal end (7) of the optical waveguide (5) and serves for coupling the narrowband light into the optical waveguide (5), and a phosphor region (19) that is present at or before the distal end (9) of the optical waveguide (5), said phosphor region being provided with a converter phosphor. The converter phosphor of the phosphor region (19) is chosen with respect to the narrowband light emitted by the narrowband light source (11) in such a way that it increases the wavelength of at least part of the narrowband light.

### BACKGROUND OF THE INVENTION

1. Field of the Invention

The present invention relates to a secondary light source and also to a medical illumination device and an optical coherence tomography device (OCT).

2. Description of the Related Art

In medical technology, so-called endoilluminators are often used in order, for example in the case of interventions on the eye, to illuminate the latter internally which is of importance particularly in the case of interventions on the rear portion of the eye. Such endoilluminators can also be used in other microsurgical or endoscopic interventions in body cavities.

It is standard practice here for the light from halogen, xenon, metal halide lamps or from high power LEDs to be coupled into optical fibers and introduced into the body cavities by means of corresponding handpieces, so called applicators. In this case, the light sources are intended to be as small as possible in order to couple the light into the optical waveguide as effectively as possible. At the distal end of the optical waveguide, the light should be emitted as far as possible uniformly and, depending on the application, directionally or diffusely into the complete space. Sometimes it is also expedient to vary the color or the color temperature of the emitted light. In traditional light sources such as halogen, xenon and metal halide lamps this can be done by means of filters. In the case of LEDs as light sources, LEDs of different colors have to be used, which makes it more difficult to couple the light into the optical fibers.

Although the hitherto customary endoilluminators based on halogen, xenon or metal halide lamps can be varied in their color by means of filters, they have a poor efficiency and generate relatively high losses in the form of waste heat. Moreover, since the emissive area is relatively large, the light can also only be coupled into the thin optical waveguides with difficulty or ineffectively.

Endoilluminators based on high-power LEDs can only be varied in their color if different-colored LEDs are used. This, too, again enlarges the emissive area and thus makes it more difficult to effect coupling into thin optical waveguides. Moreover, the emission at the distal optical waveguide end is often inhomogeneous and highly directional. In order to obtain a homogeneous emission here, which is possibly even intended to be effected into virtually the complete solid angle, special measures such as, for instance, light mixers or diffusers have to be used.

An endoilluminator for projecting illumination light into the interior of an eyeball is described in US 2004/0249424 A1, for example. It comprises a rigid hollow needle which can be used to penetrate into the eyeball. An optical fiber extends through the needle and the distal end of said optical fiber constitutes a secondary light source which can be arranged in the eyeball. A connecting piece is present at the proximal end of the fiber and can be used to connect the optical fiber to a conventional lamp as primary light source. At the present time halogen or xenon lamps are usually used as light sources. In the case of light sources of this type it is necessary to ensure sufficient dissipation of the heat generated.

Moreover, US 2004/0090796 A1 describes an endo-illuminator comprising an LED as primary light source. In comparison with halogen or xenon lamps, LEDs have a longer service life and develop less heat. However, as already mentioned, coupling LED light into the optical fiber often poses problems with regard to the efficiency and the luminous power, which adversely influences the intensity of the light at the output end of the fiber, that is to say the intensity of the secondary light source. In addition the emission at the distal end of the optical waveguide, as mentioned further above, is not optimal and therefore requires further measures, if appropriate.

JP2006261077 describes a light source comprising an LED that emits ultraviolet light, violet light, blue light or white light and an optical fiber, to the input end of which is applied a fluorescent coating composed of a converter phosphor. A color conversion of the light from the LED takes place by means of the phosphor.

Therefore, it is an object of the present invention to provide an advantageous secondary light source which can be used in particular in a medical illumination device or in an OCT.

### SUMMARY OF THE INVENTION

A secondary light source according to the invention comprises a narrowband light source that emits narrowband light as a primary radiation source, an optical waveguide having a proximal and a distal end and a coupling-in device that is arranged at the proximal end of the optical waveguide and serves for coupling the narrowband light into the optical waveguide. A phosphor region provided with a converter phosphor is present at the distal end or upstream of the distal end of the optical waveguide in the direction of the proximal end, which phosphor region can be embodied in terms of volume or area. In this case, the term “narrowband light” should also be understood to mean radiation lying just outside the spectral perception capability of the human eye, in particular light in the near ultraviolet. The converter phosphor of the phosphor region is chosen with respect to the narrowband light emitted by the narrowband light source in such a way that it increases the wavelength of at least part of the narrowband light. In particular, it can be chosen in such a way that white or broadband light emerges at the distal end of the optical waveguide. A glass fiber rod or a flexible optical fiber is suitable, in particular, as the optical waveguide. By way of example, LEDs, lasers or laser diodes can be used as narrowband light sources.

The secondary light source according to the invention involves moving away from using a white light source as primary radiation source for the radiation to be coupled into the optical waveguide. Instead, a narrowband light source is used, the narrowband light of which is coupled into the optical waveguide. It is only in the optical waveguide, and in particular shortly before or upon emerging from the optical waveguide, that the narrowband light is converted into white light or broadband light, or a color conversion is effected. For this purpose, the converter phosphor converts at least a portion of the narrowband light into light having a longer wavelength than that of the original narrowband light.

If the narrowband light coupled into the optical waveguide is visible light, part of the light coupled in can be converted into light having a longer wavelength by means of the converter phosphor, such that the superposition of the converted light with the remainder of the non-converted light originally coupled in leads to white light. By way of example, it is possible to use a narrowband light source that emits blue light. The converter phosphor can then be chosen in such a way that it converts part of the blue light into yellow light such that the superposition of the yellow light with the remaining blue light produces white light. If, by contrast, UV radiation, for example, is coupled into the optical waveguide, it is possible to convert the UV radiation completely into light in the visible spectral range by using a converter phosphor. Moreover, it is possible, by using a converter phosphor that is a mixture of different phosphors, to convert the UV radiation completely into light having at least two wavelength distributions that lead in total to white light, or into light having a broadband wavelength distribution. The use of a mixture as converter phosphor for realizing a white- or broadband-emissive secondary light source is possible, however, not only in the case of UV radiation but also when using a narrowband light source that emits visible light

A further advantage afforded by the invention is that the converter phosphor, which is generally based on fluorescence, emits a fluorescent light uniformly in all spatial directions, such that a highly homogeneous light distribution is obtained at the output end of the optical waveguide, which distribution can also additionally be influenced by means of a spatial distribution of the converter phosphor. By way of example, it is conceivable to introduce the converter phosphor in the form of small balls into the distal end of the optical waveguide.

When the secondary light source is used in endoilluminators, the latter can also be used for fluorescence applications or for photodynamic therapies if converter phosphors having corresponding emission spectra are used.

In particular through the use of a laser, a laser diode or a single LED as primary light source, in comparison with the use of lamps or white LEDs as light sources, in the secondary light source according to the invention, it is possible to optimize the coupling of the light into the optical waveguide with regard to efficiency and luminous power. An individual LED has a smaller emissive area, e.g. by comparison with an arrangement of LEDs having different colors which simplifies the coupling in. By comparison with an LED that emits white light, too, it is easier to couple in the light from an LED that emits narrowband light, since an LED that emits white light is coated with a converter phosphor that leads to emission into a large solid angle, which renders the coupling in more complicated. The use of a laser, a laser diode or a single LED as a primary light source therefore reduces the losses that occur during coupling in and thus increases the efficiency of the secondary light source.

The converter phosphor can be introduced as a doping into the phosphor region or, if the phosphor region is situated at the distal end of the optical waveguide, said converter phosphor can be applied to the distal end of the optical waveguide as a coating. In particular, phosphor regions encompassing a volume can be produced in a simple manner by means of the doping. By contrast, areal phosphor regions can be produced by coating. However, the doping of just a thin surface layer at the distal end also leads to a substantially areal phosphor region.

In particular, the converter phosphors from the group: YAG:Ce, ThAG:Ce, SrGa2S4:Eu, Ca8EuMnMg(SiO4)4C12, CaS:Eu, and SrS:Eu and mixtures of these substances are suitable for use with a narrowband light source that emits blue light. These substances are known for example, from TW 245433 B. While ThAG:Ce like YAG:Ce converts the blue light into yellow light, SrGa2S4:Eu and Ca8EuMnMg(SiO4)4C12 convert blue light into green light, and CaS:Eu and also SrS:Eu convert it into red light. The characteristic of the light emerging from the optical waveguide can thus, for example, be set in a targeted manner in particular by means of a suitable mixture of the phosphors mentioned. For the application of the secondary light source, care must be taken here to ensure the biocompatibility of the converter phosphor in particular for the case where it is applied to the distal end as a coating.

In one advantageous development of the secondary light source, a dichroic mirror coating is adjacent to the phosphor region in the direction of the proximal end of the optical waveguide. Said mirror coating is embodied such that it reflects light propagating in the direction of the proximal end. It is thereby possible to prevent light that arises in the converter phosphor from being conducted in the direction of the proximal end of the optical waveguide instead of being emitted from the distal end. The intensity of the converted light at the distal end can therefore be increased with the aid of the dichroic mirror coating in comparison with an optical waveguide not having such a mirror coating which further increases the efficiency of the secondary light source. In particular, the mirror coating can also be embodied such that it is highly reflective only for the converted light, but transmissive for the original narrowband light.

Furthermore, it is possible to arrange a partly transmissive mirror coating at the output of the distal end of the optical waveguide, which mirror coating is highly reflective for phototoxic wavelength components of the original narrowband light, but transmissive for the non-phototoxic components and the converted light. In this way, the phototoxic components can be kept away from the tissue e.g. when the secondary light source is used in an endoilluminator. At the same time, the reflected portion of the light is fed to the converter phosphor again, which increases the conversion efficiency. An almost hundred percent conversion can thus be achieved particularly in interaction with the above-mentioned mirror coating preventing the propagation of light in the direction of the proximal end of the optical waveguide.

It is particularly advantageous if a so-called monomode fiber is used as the optical waveguide. Monomode fibers are very thin optical fibers which permit only the propagation of a single oscillation mode of the electromagnetic radiation. Monomode fibers of this type can be realized with very small fiber diameters, such that, for example in the case of endoilluminators for illuminating the interior of the eyeball, it is only necessary for there to be a very small opening in the eyeball. On account of the small fiber diameter in the range of 8-10 μm, it is difficult to couple white LED light or light from thermal emitters such as lamps, for instance, in monomode fibers. The reason for this is the large solid angle into which the white LED and likewise a lamp emit. In particular lasers, but also narrowband LEDs, emit into a smaller solid angle, by contrast, since the radiation is concentrated. This is the case particularly for a laser as primary light source, such that the laser light can be coupled into optical fibers, and in particular into monomode fibers, well, for example by means of one or a plurality of lenses. The narrowband light can then be converted into white or broadband light by the phosphor region. Thus, at the exit end of the monomode fiber, white or broadband light can be emitted with an intensity that would not be achievable when using a white LED or an argon or xenon lamp.

The secondary light source according to the invention can be used for example advantageously as a light source in an endoilluminator or in other medical illumination devices, for instance in the field of endoscopy. In particular, the secondary light source according to the invention is also suitable for use in an OCT. The basic construction of an OCT is described for example in DE 199 29 406.

### BRIEF DESCRIPTION OF THE DRAWINGS

Further features, properties and advantages of the present invention will become apparent from the following description of exemplary embodiments with reference to the accompanying figures.

FIG. 1 shows an endoilluminator with a secondary light source according to the invention.

FIG. 2 shows a detail from FIG. 1.

FIG. 3 shows an alternative configuration of the secondary light source in detail.

FIG. 4 shows a further development of the secondary light source in detail.

FIG. 5 shows a modification of the further development illustrated in FIG. 4, in detail.

FIG. 6 shows an alternative endoilluminator with a secondary light source according to the invention.

FIG. 7 shows an OCT with a secondary light source according to the invention.

FIG. 8 shows the secondary light source of the OCT from FIG. 6.

FIG. 9 shows a spectrum of the light emitted by the secondary light source.

### DETAILED DESCRIPTION OF THE PREFERRED EMBODIMENT

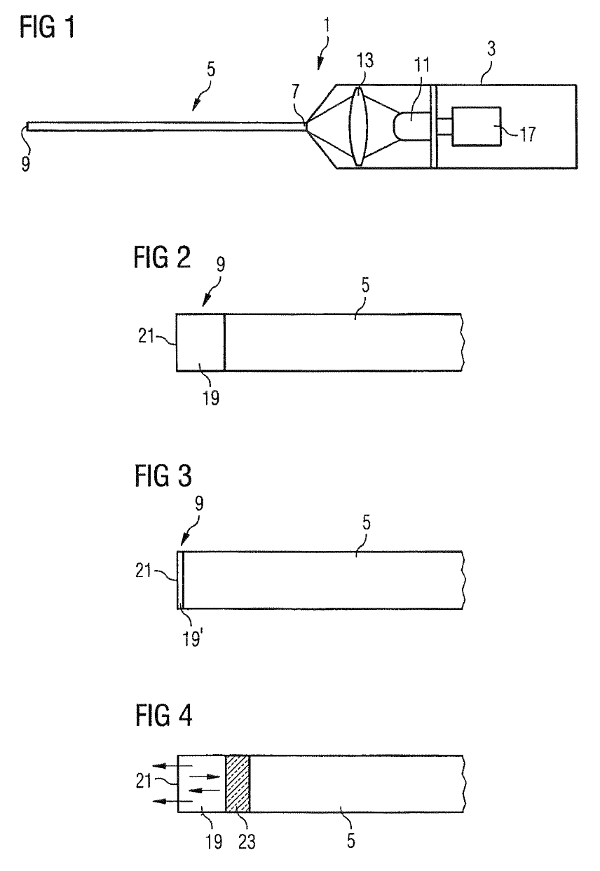

FIG. 1 shows an endoilluminator 1 in a highly schematic illustration as an exemplary embodiment of a medical illumination device comprising a secondary light source according to the invention. The endoilluminator 1 comprises a handle 3 and a glass fiber rod 5. The glass fiber rod 5 serves as an optical fiber having a proximal end 7, into which is coupled light from a primary light source 11 arranged in the interior of the handle 3, and a distal end 9, to which the light coupled in is guided and from which said light emerges.

In the present exemplary embodiment, the glass fiber rod is embodied as a monomode fiber. The diameter of the glass fiber rod is therefore very small (8-10 μm), such that it can be inserted through just small openings into the body, whereby trauma can be minimized.

Although not explicitly illustrated in the figures, the glass fiber rod 5 is surrounded by a sheath, which can likewise be thin. The latter additionally can be surrounded by a protective sleeve, which, in particular, can also be sterilizable.

As illustrated in FIG. 1, the glass fiber rod 5 can be fixedly connected to the handle 3. As an alternative, however, at its proximal end 7, it can also have an optical plug connection that can interact with a corresponding optical plug connection at the handle 3 for connecting the glass fiber rod to the handle 3. Such a plug connection would enable the glass fiber rod 5 to be exchanged in a simple manner.

There is additionally present in the handle 3 an electronic unit 7 for suitably supplying the primary light source 11 with voltage. Furthermore, a power source, for example a rechargeable battery, can also be integrated into the handle 3. As an alternative, it is possible to connect the handle 3 to an external power supply, for example, a public mains supply system, by means of an electrical line (not illustrated).

The primary light source is a laser diode 11 in the present exemplary embodiment. The light emitted by the laser diode 11 is coupled into the proximal end 7 of the glass fiber rod 5 by a coupling-in device, which is indicated by a lens 13 in FIG. 1. Although it is indicated by a lens 13, the coupling-in device can also comprise mirrors and other optical elements in addition to lenses or as an alternative to lenses. A laser diode 11 that emits blue laser light is used in the endoilluminator 1 illustrated. The blue laser light is coupled, by means of the coupling-in device 13, into the proximal end 7 of the glass fiber rod 5, from where it is guided to the distal end 9.

FIG. 2 shows a detailed illustration of the distal end 9 of the glass fiber rod 5. A phosphor region 19, in which the glass fiber material is doped with YAG:Ce, is situated at the distal end 9. YAG:Ce forms a converter phosphor that converts part of the blue laser light arriving at the distal end 9 into yellow light. In this case, the YAG:Ce concentration in the phosphor region 19 is chosen in such a way that the proportion of blue light converted into yellow light has a magnitude precisely such that the mixture of blue and yellow light upon emerging from the distal end 9 appears substantially white. The larger the axial extent of the phosphor region 19 in the distal end of the glass fiber rod, the lower the concentration of the YAG:Ce need be.

As an alternative to the YAG:Ce, the phosphor region 19 can contain ThAG:Ce as the converter phosphor. If the secondary light source is intended to effect particularly broadband emission, the converter phosphor can contain a mixture of at least two of the following substances: YAG:Ce, ThAG:Ce, SrGa2S4:Eu, Ca8EuMnMg(SiO4)4C12, CaS:Eu or SrS:Eu.

The doping can be effected, for example, by the corresponding substance being applied to the surface of the phosphor region 19 and said surface subsequently being subjected to a thermal treatment during which the substance diffuses into the glass fiber material. The dopant can also be introduced by means of implantation. In this case, the implantations can be supported, if appropriate, by a subsequent thermal treatment during which diffusion of the implanted dopant takes place.

An alternative configuration of the phosphor region is illustrated in FIG. 3. In this variant, at the distal end 9 of the glass fiber rod 5 unlike in FIG. 2 the phosphor region 19′ is not embodied in a manner encompassing a volume, but rather in areal fashion. The areal phosphor region 19′ is formed by a coating applied to the exit end 21 of the glass fiber rod 5. The coating can be applied by all suitable coating methods, for instance by means of chemical or physical vapor deposition (CVD or PVD). The coating comprises YAG:Ce in the present exemplary embodiment. However, it can in principle comprise the same substances as were described with regard to the phosphor region 19 encompassing a volume from FIG. 2.

Although the areal phosphor region 19′ is realized in the form of a coating in the present exemplary embodiment, it can in principle also be produced by a shallow doping, i.e. doping near the surface.

A further development of the secondary light source whose distal end is illustrated in FIG. 2, is shown in FIG. 4. Like FIG. 2, FIG. 4 shows the distal end 9 of the glass fiber rod 5 with the phosphor region 19 encompassing a volume that is present there. In contrast to the variant illustrated in FIG. 2, in the variant illustrated in FIG. 4, a dichroic mirror coating 23 is adjacent to the phosphor region 19 in the direction of the proximal end 7 of the glass fiber rod 5. Said mirror coating is configured such that it allows the blue light coming from the proximal end to pass through without any disturbance. By contrast, the yellow light arising in the phosphor region 19 is reflected. Therefore, yellow light propagating from the phosphor region in the direction of the proximal end of the glass fiber rod 5 is reflected in the direction of the distal end 9 of the glass fiber rod 5 by the dichroic mirror coating. What is achieved in this way is that yellow light which propagates in the direction of the proximal end 7 after emission also contributes to the illumination, with the result that the yield of white light at the distal end 9 of the glass fiber rod 5 is optimized and the secondary light source has an optimal intensity.

It goes without saying that the dichroic mirror coating can also be used in the case of a coated phosphor region 19′ instead of in the case of a doped phosphor region 19. Moreover, instead of the glass fiber rod, a flexible optical fiber can be present as the optical waveguide.

FIG. 5 illustrates a modification of the distal end 9 of the glass fiber rod 5 illustrated in FIG. 4. In this modification, there is a second dichroic mirror coating 25 present at the exit end 21 of the glass fiber rod 5. This second dichroic mirror coating 25 reflects the not-converted blue light coming from the laser diode back in the direction of the proximal end 7 of the glass fiber rod 5. It is not necessary that the entire spectral range of blue light is reflected to the proximal end 7. Rather, it is sufficient if at least the phototoxic wavelength components of said blue light are reflected back. Moreover, the dichroic mirror coating 23 situated upstream of the exit end 21 at a distance in the direction of the proximal end 7 can be configured in this modification in such a way that it reflects not only the yellow light arising in the phosphor region 19 but also blue light propagating from the exit end 21 in the direction of the proximal end 7 of the glass fiber rod 5. Said blue light is then reflected back and forth between the mirrors 23, 25 until it is completely converted by the converter phosphor. In this case, the secondary light source of the endoilluminator emits yellow light having a broad spectral distribution instead of white light.

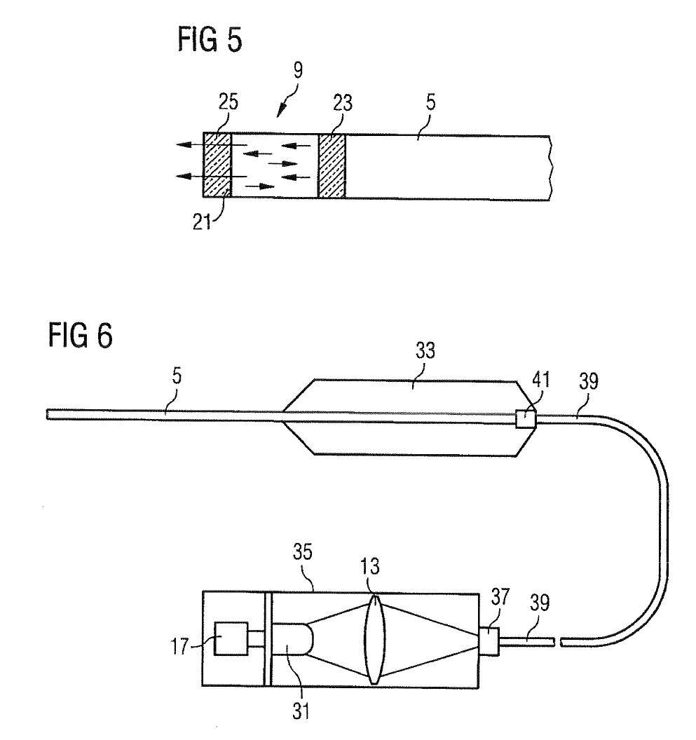

A modification of the endoilluminator described with reference to FIG. 1 is illustrated in FIG. 6. In order to avoid repetition, elements corresponding to elements in FIG. 1 are designated by the same reference numerals as in FIG. 1 and are not described again.

In contrast to the embodiment variant of the endoilluminator as illustrated in FIG. 1, the glass fiber rod 5 in the variant shown in FIG. 6 is only surrounded by a simple gripping piece 33 through which the glass fiber rod 5 extends. The laser diode 31, the coupling-in device 13 and the electronic unit 17 are arranged in a separate supply unit 35. The latter is equipped with a receptacle for an optical plug connector 37, into which can be plugged a corresponding optical plug connector at the proximal end of an optical fiber 39. The distal end of said optical fiber 39 can be plugged into an optical plug connector 41 on the handle 33. The optical plug connector 41 is designed for transmitting the light that emerges from the optical fiber 39 into the glass fiber rod 5 extending to the gripping piece 33.

The light source used in the present exemplary embodiment is a solid-state laser 31 that emits blue or violet light having a wavelength of below 420 μm. The configuration in accordance with FIG. 6 enables the gripping piece 33 and the elements of the endoilluminator that are to be handled by means of the gripping piece 33 to be made particularly light. Moreover, it is thus also possible to use electronic units, coupling-in devices or lasers which would be too bulky or heavy for integration into the handle. Finally, this configuration also makes it possible to couple a plurality of optical fibers 39 to a common supply unit 35, such that a plurality of glass fiber rods 5 can be supplied with laser radiation from a powerful laser by the same supply unit 35.

The distal end of the glass fiber rod 5 corresponds to the distal end of the endoilluminator described with reference to FIGS. 1 to 5. As an alternative, however, it is also possible to shift the phosphor region into the optical fiber 39 between supply unit 35 and handle. In this case, the exit end of the optical fiber 39 rather than the exit end of the glass fiber rod 5 forms the secondary light source according to the invention.

Both the glass fiber rod 5 and the optical fiber 39 can be embodied as monomode fibers, in particular.

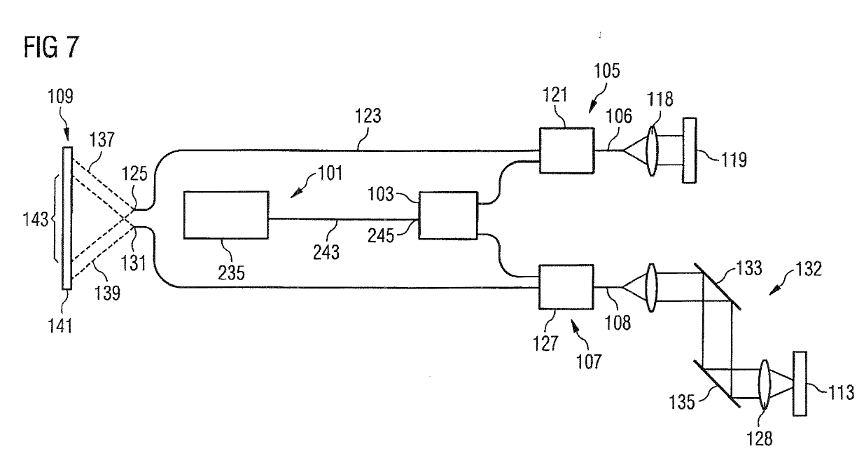

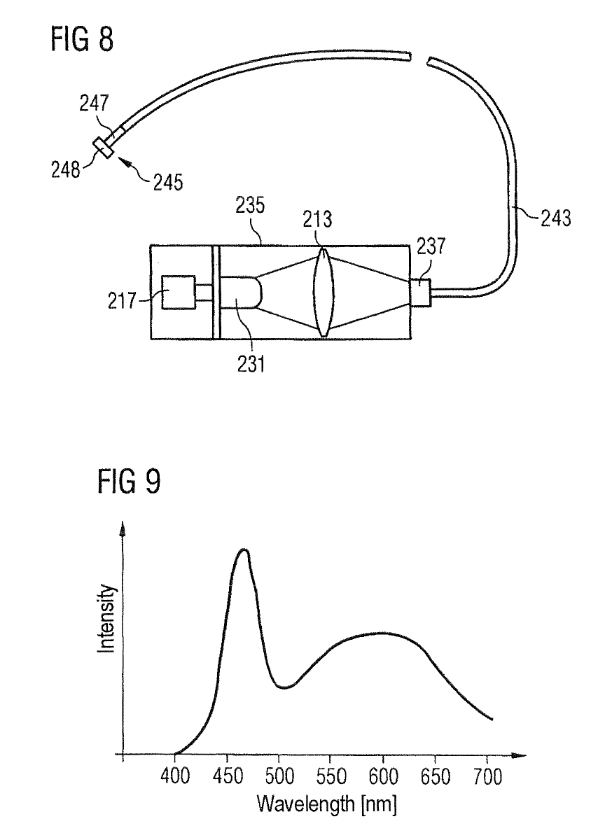

An OCT device comprising a secondary light source according to the invention is described below with reference to FIGS. 7 and 8.

The OCT device comprises a secondary light source 101 according to the invention for emitting temporally incoherent light, a splitter 103 for splitting the light into a reference beam and a measurement beam, a reference branch 105, into which the reference beam is coupled by the splitter 103 and in which said reference beam covers a defined distance, a measurement branch 107, into which the measurement beam is coupled by the splitter 103 and via which the measurement beam is fed to a sample 113, and also a detector 109, in which the measurement light reflected from the sample 113 is superposed with reference light from the reference branch 105 and the superposed light is detected.

The secondary light source 101 is a broadband light source that emits essentially temporally incoherent radiation. It is illustrated in detail in FIG. 8 and comprises a supply unit 235, which corresponds to the supply unit 35 from FIG. 6 and is therefore not described again in detail. An optical fiber 243 is connected to the optical plug connector 237 of the supply unit 235, the distal end 245 of said optical fiber being provided with a phosphor region 247 as was described with reference to FIG. 2. Moreover, the distal end 245 of the optical fiber 243 has an optical plug connector 248 that can be plugged into the mixer 103.

It goes without saying that the distal end 245 of the optical fiber 243 can also be provided with the dichroic mirror coating described with reference to FIG. 4 or FIG. 5. It is likewise possible to form the phosphor region 247 at the distal end 245 by means of a coating instead of by doping. The coated phosphor region also can be provided with a dichroic mirror coating that is adjacent in the direction of the proximal end of the optical fiber 243.

A spectral distribution of the light from the secondary light source 101, that is to say that the light that emerges from the distal end 245 of the optical fiber 243, when using YAG:Ce as a converter phosphor, is illustrated in FIG. 9. The distribution has a relatively narrow line in the blue spectral range with a central wavelength of approximately 460 μm and a broad distribution with a central wavelength at approximately 550 μm and a width of approximately 100 μm. In this case, the blue spectral line of the spectrum in FIG. 9 represents the unconverted blue light from the laser diode 231. It should be noted that this blue spectral line is not a line in the strict sense, but is broadened as shown in FIG. 9. The spectral location of the blue line shown in FIG. 9 is given by the maximum of the peak. The white light emitted by the secondary light source 101 is thus broadband enough for use in an OCT device. The coherence length of the light source 101 determines the depth resolution of the OCT. The coherence length of the laser light can be reduced by pulsed operation of the laser. This spectral distribution is also present in the exemplary embodiment described with reference to FIGS. 1 to 5 for an endoilluminator comprising a secondary light source according to the invention. The blue line is absent, however, in the modification described with reference to FIG. 5.

In the reference branch 105, the reference light beam is coupled into a reference optical waveguide 106 and fed to a mirror 119 via an optical unit 118. The mirror 119 reflects the reference beam, which is coupled into the reference optical waveguide 106 again after reflection by the optical unit 118. A mixer 121 mixes the reflected reference light with the reference beam coming from the splitter 103 in a ratio of 50:50 and couples the light conditioned in this way into a further reference optical waveguide 123, which leads to the detector 109 and which guides the reference light beam to a beam output 125 of the reference branch 105. The reference optical waveguides and all the other optical waveguides are preferably monomode fibers.

The measurement light is fed via a measurement optical waveguide 108 arranged in the measurement branch 107 to a scanning device 132, by which it is directed on to an optical unit 128 that focuses the measurement light beam on to a sample region. The scanning device 132 comprises a first galvanometer mirror 133, which can be pivoted about an axis, for imparting an X deflection of the measurement beam and also a second galvanometer mirror 135 which can be pivoted about an axis, for imparting a Y deflection of the measurement beam. The axes about which the respective galvanometer mirrors 133, 135 can be pivoted are preferably perpendicular to one another, but can also assume any desired angles with respect to one another as long as they are not parallel to one another. By means of a scanning controller (not illustrated), the galvanometer mirrors 133, 135 are controlled in such a way that a specific sample region is scanned step by step. In each scanning step, the light reflected by the sample 113 is in this case picked up by the microscope optical unit 128 and fed to the measurement optical waveguide 108 again via the scanning device 132. Scanning devices other than the one described alternatively can be used.

A mixer 127, to which the measurement light is conducted via the measurement optical waveguide 108, mixes the measurement light reflected by the sample in a ratio of 50:50. The measurement light conditioned in this way is coupled by the mixer 127 into a further measurement optical waveguide 129, likewise preferably a monomode fiber, which conducts the measurement light to the beam output 131 of the measurement branch 107.

From the beam outputs 125, 131 of the reference branch 105 and of the measurement branch 107, respectively, the reference light and the measurement light are directed in the form of light cones 137, 139 onto a CCD line 141 of the detector 109, which CCD line represents the sensor area of the detector 109. The two beam outputs 125, 131 are arranged at a distance from one another, such that the two light cones are partly superposed and simultaneously illuminate at least one partial region 143 of the CCD line 141. Interference phenomena occur only if the measurement light arriving at one point of the CCD line 141 has covered the same distance as the reference light arriving at the same point of the CCD line 141. From the known path lengths which the reference light has to cover from the beam output 125 to the respective points on the CCD line, a depth within the sample 113 can be assigned to the respective point on the CCD line 141. Only measurement light that was reflected at said depth interferes with the reference light at the assigned point of the CCD line 141. A read-out unit (not illustrated) reads the CCD line and forwards the data read out to an evaluation unit (likewise not illustrated), which performs the assignment of a pixel to the sample depth from which the measurement light impinging on the pixel originates.

What is claimed is:

1. Secondary light source comprising:
a narrowband light source (11, 31, 231) that emits narrowband light as a primary light source,
an optical waveguide (5, 243) having a proximal and a distal end (9, 245)
a coupling-in device (13, 213) that is arranged at the proximal end (7) of the optical waveguide (5, 243) and serves for coupling the narrowband light into the optical waveguide (5, 243), and
a phosphor region (19, 19′, 247) that is present at or before the distal end (9, 245) of the optical waveguide (5, 243), said phosphor region being provided with a converter phosphor,
wherein the converter phosphor of the phosphor region (19, 19′, 247) is chosen with respect to the narrowband light emitted by the narrowband light source (11, 31, 231) in such a way that it increases the wavelength of at least part of the narrowband light.

2. Secondary light source according to claim 1, in which the converter phosphor of the phosphor region (19, 19′, 247) is chosen with respect to the narrowband light emitted by the narrowband light source (11, 31, 231) in such a way that white or broadband light emerges at the distal end (9, 245) of the optical waveguide.

3. Secondary light source according to claim 1, in which the converter phosphor is introduced as a doping into the phosphor region (19, 247) of the optical waveguide (5, 243).

4. Secondary light source according to claim 1, in which the phosphor region (19′) is situated at the distal end (9) of the optical waveguide (5) and the converter phosphor of the phosphor region (19′) is applied to the distal end (9) of the optical waveguide (5) as a coating.

5. Secondary light source according to claim 1, in which a dichroic mirror coating (23) is adjacent to the phosphor region (19) in the direction of the proximal end (7) of the optical waveguide.

6. Secondary light source according to claim 5, in which a dichroic mirror coating (25) is present at the exit end (21) of the optical waveguide.

7. Secondary light source according to claim 1, in which the optical waveguide is a glass fiber rod (5).

8. Secondary light source according to claim 1, in which the optical waveguide (5, 243) is a monomode fiber.

9. Secondary light source according to claim 1, in which the narrowband light source (11, 31, 231) emits blue light and the converter phosphor is chosen in such a way that the blue light is converted into white light or broadband light.

10. Secondary light source according to claim 1, in which the converter phosphor is selected from the group YAG:Ce, ThAG:Ce, SrGa₂S₄:Eu, Ca₈EuMnMg(SiO₄)₄C₁₂, CaS:Eu, SrS:Eu or mixtures thereof.

11. Secondary light source according to claim 1, in which the narrowband light source is a diode (11).

12. Secondary light source according to claim 1, in which the narrowband light source is a laser (11).

13. Medical illumination device comprising:
a secondary light source comprising:
a narrowband light source (11, 31, 231) that emits narrowband light as a primary light source,
an optical waveguide (5, 243) having a proximal and a distal end (9, 245)
a coupling-in device (13, 213) that is arranged at the proximal end (7) of the optical waveguide (5, 243) and serves for coupling the narrowband light into the optical waveguide (5, 243), and
a phosphor region (19, 19′, 247) that is present at or before the distal end (9, 245) of the optical waveguide (5, 243), said phosphor region being provided with a converter phosphor,
wherein the converter phosphor of the phosphor region (19, 19′, 247) is chosen with respect to the narrowband light emitted by the narrowband light source (11, 31, 231) in such a way that it increases the wavelength of at least part of the narrowband light.

14. Medical illumination device according to claim 13, which is configured as an endoilluminator (1).

15. Optical coherence microscope comprising:
a secondary light source comprising:
a narrowband light source (11, 31, 231) that emits narrowband light as a primary light source,
an optical waveguide (5, 243) having a proximal and a distal end (9, 245)
a coupling-in device (13, 213) that is arranged at the proximal end (7) of the optical waveguide (5, 243) and serves for coupling the narrowband light into the optical waveguide (5, 243), and
a phosphor region (19, 19′, 247) that is present at or before the distal end (9, 245) of the optical waveguide (5, 243), said phosphor region being provided with a converter phosphor,
wherein the converter phosphor of the phosphor region (19, 19′, 247) is chosen with respect to the narrowband light emitted by the narrowband light source (11, 31, 231) in such a way that it increases the wavelength of at least part of the narrowband light.
